(19) United States
(12) Patent Application Publication
(10) Pub. No.: US 2024/0016658 A1
(43) Pub. Date: Jan. 18, 2024

(54) NON-GRAVITATIONAL FLUID DELIVERY DEVICE FOR OPHTHALMIC APPLICATIONS

(71) Applicant: Twenty Twenty Therapeutics LLC, South San Francisco, CA (US)

(72) Inventor: Timothy Stowe, South San Francisco, CA (US)

(21) Appl. No.: 18/351,129

(22) Filed: Jul. 12, 2023

Related U.S. Application Data
(60) Provisional application No. 63/389,132, filed on Jul. 14, 2022.

(51) Int. Cl. A61F 9/00; A61L 2/10

(52) U.S. Cl. CPC A61F 9/0026; A61L 2/10; A61L 2202/24

(57) ABSTRACT

Aspects of the present disclosure include fluid dispensers configured to administer pharmaceutical agents to a user's eye. The fluid dispensers and systems described herein include mechanisms for protecting a fluid dispensing nozzle or opening from contamination, preventing dehydration of the fluids, and for sterilizing the dispensing components. A fluid dispenser may include a housing configured to receive a fluid cartridge, the fluid cartridge comprising a nozzle. The housing further comprises a shutter coupled to the housing and configured to articulate between a closed position and an open position. The shutter may be configured to obscure the nozzle in the closed position and to expose the nozzle in the open position. The shutter may further include protrusion configured to engage and actuate a nozzle cover of the fluid cartridge when the shutter articulates to the open position.

### CROSS-REFERENCE TO RELATED APPLICATIONS

The present application claims the benefit of and priority to U.S. Provisional Patent Application No. 63/389,132, filed Jul. 14, 2022, the entirety of which is incorporated by reference herein.

### TECHNICAL FIELD

The present disclosure relates in general to a device to deliver therapeutic fluids to an eye of a user. The device allows for the non-gravitational delivery of viscous ophthalmic drugs to the eye using one or more micro-streams.

### BACKGROUND

Many eye-drop medications and artificial tear formulations with increased formulation viscosity (e.g., 50 centipoise (cps) to 200 cps) have been shown to have longer residence time, better mucosal adhesion (adhesion to mucin cells), and improved corneal hydration. This is important for dry eye diseases but also important for other drug delivery applications where higher concentrations and longer residence time improve drug delivery efficacy.

Dispensing higher viscosity fluids (e.g., fluids having a viscosity of between 50 cps to 200 cps) from conventional eye droppers is not ideal for a number of reasons. First, the dose volume for a conventional eye dropper varies. The dose volume can range anywhere from 30 to 65 μL, with a repeatability of about +/−5 μL or about +/−10% of standard deviation. The tilt angle range, which people use during application using a conventional eye dropper, can have a measurable impact on drop volume by up to an additional 10% variability on top of the standard deviation and dose volume range set forth above. To account for partial misses of fluid delivery to the eye, an excess of fluid is typically delivered to the eye using a conventional eye dropper. When the dose volume varies and there is an excess fluid applied to the eye, the excess fluid sometimes takes several minutes to drain from the eye, which can temporarily lead to a non-uniform tear layer that causes blurring due to spherical and comb aberrations. A further nuisance is that sometimes the excess viscous drop volume partially misses the eye during application and then gets stuck in the eyelashes, which leads to crusting as the drop dries out.

Second, the shape and size of a drop resulting from a conventional eye dropper results in reduced uniform spreading of the drop over the eye. Generally, a conventional shape and size of a 50 μL drop resulting from a conventional eye dropper is a semi-sphere having a diameter approximately 5 mm. When a 5 mm diameter sphere contacts the eye, there is approximately about 2 mm of margin on either side of the drop between the drop and the eyelid. As such, it is often difficult to hit the eye without a portion of the drop landing or splashing outside of the eye. When the drop is of a highly viscous fluid, the drop that hits the corneal surface can be approximately 2-3 mm in height as measured normal to the surface of the cornea. The wiping action of the human eyelid does not do well to force the uniform spreading of such a tall perturbation given the eyelid itself is only approximately 3-4 mm thick. As such, uniform spreading becomes more challenging with high viscosity formulations.

Accordingly, for high viscosity formulations, it is preferred to dispense smaller, uniform doses across the eye and allow the eyelids to spread the small drops uniformly in the vertical direction (e.g., between eyelids). Using smaller doses reduces or eliminates problems associated with short term blurring and can allow for even higher viscosity formulations that are more effective in terms of their residency time and moisture retention, and therefore, are more pleasing to the end user.

Moreover, with conventional reusable eye drop systems, preservatives are often included in the dispensed fluid to prevent the growth of bacterial or viral germs. These preservatives may result in damage and corneal sensitization over time for those people that regularly use the drops. While a filter may be used to reject the preservative before it reaches the eye of the user, the filter may not be applicable to all types of fluids/formulations. Multidose preservative-free eye dropper systems do not include preservatives but require built-in filters and unidirectional valves. These elements add complexity and significant cost to the packaging of the reusable eye drop system.

Finally, conventional reusable eye drop systems may be susceptible to bacterial and/or viral contamination especially at the outside of the tip which can come in direct contact with eyes and also with bathroom countertops. For example, contamination in and/or around a dispensing nozzle of a conventional eye drop system may travel with a dispensed drop into the patient's eye, causing infection.

### SUMMARY

Aspects of the present disclosure include fluid dispensers configured to administer pharmaceutical agents to a user's eye. The fluid dispensers and systems described herein include mechanisms for protecting a fluid dispensing nozzle or opening from contamination, preventing dehydration of the fluids, and for sterilizing the dispensing components. For example, according to one embodiment, a fluid dispenser includes a housing defining: a first opening configured to receive a fluid cartridge; and a second opening positioned such that a nozzle of the fluid cartridge is aligned with the second opening when the fluid cartridge is positioned within the first opening. The fluid dispenser further includes a shutter coupled to the housing and configured to articulate between a closed position and an open position. The housing shutter may be configured to obscure the second opening in the closed position and to expose the second opening in the open position. In some embodiments, the shutter comprises a protrusion on an inward-facing surface of the shutter, the protrusion configured to engage and actuate a nozzle cover of the fluid cartridge when the shutter articulates to the open position. The housing shutter may protect the internal nozzle cover from dust and debris when the cartridge is not in use and can protect externally facing sensor elements such as a blink detection or proximity sensor from dust and debris as well which are not part of the fluid cartridge.

In some embodiments, the shutter includes a first shutter component and a second shutter component coupled to the first shutter component by a first hinge. In some embodiments two or more normally separate parts of the shutter mechanical system could be connected by a living hinge being made of a thin connected flexible polypropylene membrane that can be repeatedly and reliably flexed. Thus cost can be saved if multiple rotating elements can be premade and assembled as one part. In some embodiments, the fluid dispenser further includes a mechanical motor actuator coupled to the second shutter component by a second hinge, wherein the motor actuator is configured to rotate to move the shutter from the closed position to the open position via a worm gear. In some embodiments, the fluid dispenser further includes a sterilization component coupled to the first shutter component. In some embodiments, the fluid dispenser further includes: a sterilization component coupled to the inward-facing surface of the shutter, where the sterilization component is configured to emit light toward the nozzle of the fluid cartridge when the shutter is in the closed position and the fluid cartridge is positioned within the first opening. In some embodiments, the fluid dispenser further includes: a pair of conductors coupled to the shutter and the sterilization component; and a pair of electrical contacts coupled to the housing. In some embodiments, the pair of electrical contacts are positioned to be in contact with the pair of conductors when the shutter is in the closed position. In some embodiments, the fluid dispenser further includes a power supply coupled to the housing and configured to provide electrical power to the sterilization component via the pair of electrical contacts. In some embodiments, the fluid dispenser further includes a controller configured to cause the power supply to provide the electrical power to the sterilization component for one or more periods of time.

According to another embodiment of the present disclosure, a fluid dispensing system may include a fluid cartridge comprising: a reservoir configured to retain a fluid; a dispensing head comprising a nozzle; and a polymeric external nozzle cover movably coupled to the dispensing head. The fluid dispensing system may further include an actuator assembly comprising a housing configured to releasably retain the fluid cartridge. The housing may include: a first opening configured to receive the fluid cartridge; and a second opening positioned adjacent the nozzle of the fluid cartridge when the fluid cartridge is disposed within the first opening. The fluid dispensing system may further include a shutter coupled to the housing and configured to articulate between a closed position and an open position. In some embodiments, the shutter is configured to actuate the polymeric nozzle cover to expose the nozzle when the shutter is articulated from the closed position to the open position. In some embodiments, the shutter comprises an inward-facing surface, wherein the inward-facing surface faces the nozzle when the shutter is in the closed position. The fluid dispensing system may further include a sterilization component coupled to the inward-facing surface of the shutter.

In some embodiments, the dispensing head further includes a compression membrane on a first side of the dispensing head, and wherein the drop actuator assembly further comprises a solenoid configured to strike the compression membrane to expel fluid through the nozzle. In some aspects it may be advantageous for the solenoid to be of a bi-stable or two position latching type. A bi-stable solenoid or a latching-type solenoid may maintain both the latched and unlatched positions without constant current draw so that current is supplied only during the transition from one state to another. In addition, the force and speed and momentum of the movement with which the plunger strikes may be adjusted electronically in solenoid designs by varying the amount of current supplied. Thus higher de-latching current may provide a higher strike force for higher viscosity liquids formulations inside the cartridge to maintain a dosage for a variety of fluid viscosities. It may be advantageous for the solenoid to reduce or eliminate rebound so as not to resonate back and forth or bounce as this can draw outside air inside the nozzle opening during immediate retraction or bounce thus preventing or hindering the potential use of preservative-free formulations. Thus a solenoid with a damping feature such as a soft elastic dampener to eliminate rebound motion may be used. Combining the dampener with a bistable operation may allow for the outward push state to be maintained at the end of travel without any immediate retractions when current is turned off. The compression membrane can act as an internal elastic nozzle cover as well as dampener on the inside of the nozzle blocking penetration of external pathogens. For eliminating such unwanted ingress, the external nozzle cover may be changed to the closed position before the internal compression membrane is pulled away to return to its open or retracted position by moving the solenoid from an outward pushed state to a retracted pulled state. Thus a bistable solenoid with adjustable strike force having an end point dampener may be an advantageous configuration for a micro stream ejecting actuator.

To coordinate the opening and closing of the nozzle cap with the shutter opening in the housing it may be advantageous to include a coupling feature between these two elements. In some embodiments, the fluid cartridge further comprises an arm coupled to the polymeric cover, where the arm is movable between an open state and a closed state, and where the nozzle is exposed when the arm is in the open state. In some embodiments, the shutter includes a nozzle cover disengagement feature configured to actuate the arm to the open state. In some embodiments, the arm is spring-biased such that the polymeric cover is in the closed state when the shutter is in the closed position. The spring can provide extra sealing pressure on the external nozzle cap when disengaged for a tighter seal. In some embodiments, the fluid dispensing system may further include a mechanical actuator coupled to the shutter, where the mechanical actuator is configured to actuate the shutter to move from the closed position to the open position. In some embodiments, the mechanical actuator is configured to actuate the polymeric cover to move from the closed state to the open state.

To eliminate and lower the ingress of any outside bacterial pathogens for preservative free formulations, the fluid dispensing devices and systems described herein may be configured with brief times of exposure to the external environment during ejection of micro stream drops of material. In some aspects, pathogens, even those with self-locomotion mechanisms like flagella, may have limited mobility and may not travel significant distances upstream in a short time period. Accordingly, in this time period it may be possible to sterilize these pathogens provided sufficient sterilization dosage can be provided. With the advent of improved efficiency and low cost short wavelength LED sources such sterilization approaches are now a viable option.

In some embodiments, the fluid dispensing system further includes: a pair of conductors coupled to the shutter and the sterilization component; a pair of electrical contacts coupled to the housing, where the pair of electrical contacts are positioned to be in contact with the pair of conductors when the shutter is in the closed position; and a power supply coupled to the housing and configured to provide electrical power to the sterilization component via the pair of electrical contacts. In some embodiments, the fluid dispensing system further includes a controller configured to cause the power supply to provide the electrical power to the sterilization component for one or more periods of time.

According to another embodiment of the present disclosure, a fluid dispensing device includes a housing defining an opening, where the housing comprises a pair of electrical contacts adjacent to the opening. The fluid dispensing device may further include a shutter assembly movably coupled to the housing, where the shutter assembly includes a first shutter component; and a mechanical motorized or hand-triggered actuator coupled to the housing and the first shutter component. In some embodiments, the mechanical actuator is movable from a first position to a second position, where the mechanical actuator is configured to cause the shutter assembly to move from an open position to a closed position when the mechanical actuator moves from the first position to the second position. In some embodiments, the fluid dispensing device further includes a sterilizing subassembly coupled to the first shutter component, where the sterilizing subassembly comprises: a sterilizing light element; and a pair of electrical conductors electrically coupled to the sterilizing light element. In some aspects, the pair of electrical conductors are configured to contact with the pair of electrical contacts when the shutter assembly is in the closed position.

In some embodiments, the mechanical actuator is coupled to the first shutter component via a second shutter component, wherein the first shutter component is rotatably coupled to the second shutter component by a hinge. In some embodiments, the first shutter component is positioned to obscure the opening when the shutter assembly is in the closed position. In some embodiments, the housing comprises a track feature, and the first shutter component comprises a guide feature positioned at least partially within the track feature. In some embodiments, the guide feature is configured to slide within the track feature when the shutter assembly moves from the open position to the closed position.

It will be understood that the embodiments and aspects described above are exemplary and that additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a cross-sectional view of an example non-gravitational fluid delivery system in accordance with at least one embodiment of the present disclosure, the non-gravitational fluid delivery device system including a fluid cartridge housed within a dispensing device.

FIG. 2A is a cross-sectional view of the fluid cartridge and fluid dispensing device of FIG. 1 with a shutter assembly in a closed position, in accordance with at least one embodiment of the present disclosure.

FIG. 2B is a cross-sectional view of the fluid cartridge and fluid dispensing device of FIG. 1 with the shutter assembly in an open position, in accordance with at least one embodiment of the present disclosure.

FIG. 3 is a cross-sectional view of a fluid cartridge and fluid dispensing device, in accordance with at least one embodiment of the present disclosure.

FIG. 4A is a perspective view of a shutter assembly and a sterilization component of a fluid dispensing device, in accordance with at least one embodiment of the present disclosure.

FIG. 4B is a perspective view of a shutter assembly of a dispensing device and a nozzle cover of a fluid cartridge, in accordance with at least one embodiment of the present disclosure.

FIG. 5A a perspective view of a dispensing device including sterilization components for sterilizing a fluid cartridge nozzle, in accordance with at least one embodiment of the present disclosure.

FIG. 5B a front elevation view of the dispensing device shown in FIG. 5A, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Disclosed herein is one example of a non-gravitational dropper device and/or sprayer device that delivers a fluid to a patient or user. However, neither the term “spray”, “sprayer”, “drop”, or “dropper” are limiting, as the fluid that is dispersed from the device may be considered a “stream”, “micro-stream”, or “sheet” of fluid. Generally, the fluid dispersed from the device includes a stream of liquid. Generally, the device delivers a fluid to the eye of a patient, but the device could be used for other applications, such as to deliver viscous fluid medications to the nose or mouth in other applications.

Aspects of the present disclosure include non-gravitational ophthalmic fluid dispensing devices and systems. A non-gravitational ophthalmic fluid delivery device may be configured to deliver a therapeutic fluid in a horizontal configuration. For example, a fluid dispensing system may be configured to produce a jet or stream of fluid with sufficient velocity to travel to the patient's eye in the horizontal configuration. The fluid dispensing system may include a fluid cartridge coupled to or disposed within a fluid dispensing device. The fluid cartridge may be a reusable cartridge, or may be a single-use cartridge. In some embodiments, the fluid cartridge may include a single-use fluid reservoir coupled to a reusable dispensing head. In other embodiments, both the fluid reservoir and the dispensing head are single-use.

As mentioned above, conventional eye droppers may be susceptible to contamination from bacteria, viruses, and/or other types of contamination. The contamination may be carried by the fluid administered in the patient's eye. Accordingly, it may be advantageous to periodically sterilize the nozzle and/or dispensing area of a fluid applicator. Further, it may be advantageous to provide for automatic nozzle sterilization that does not involve or require patient compliance.

Aspects of the present disclosure described fluid delivery systems, devices including one or more sterilizing components for sterilizing the dispensing nozzle and/or nozzle area. Further aspects of the present disclosure allow for automatic and/or periodic sterilization of the nozzle and nozzle area based on the open or closed position of the fluid dispensing device. For example, embodiments of the present disclosure may include a fluid dispensing device having a door or shutter assembly configured to move between an open position and a closed position. In the open position, the door or shutter assembly is retracted to expose the nozzle through an opening in the fluid dispensing device. Accordingly, in the open position, the fluid dispensing device may be ready for use. In the closed position, the door or shutter assembly may be close to obscure, protect, and/or otherwise isolate the nozzle from the external environment. For example, after a user has finished using the fluid delivery device, the user may use a mechanical and/or electrical actuator or trigger to move the shutter assembly to the closed state to prevent potential contamination or damage to the nozzle area. Sterilization components may be coupled to the shutter assembly, directly or indirectly. The sterilization components may be configured to sterilize the nozzle and/or nozzle area based on the shutter assembly changing to the closed position. The sterilization components may be activated for a single period of time or for multiple periods of time in response to the shutter assembly moving to the closed position. Because the shutter assembly may be an integral component of the fluid dispensing device, it may be unlikely for the sterilization components to be lost or misplaced.

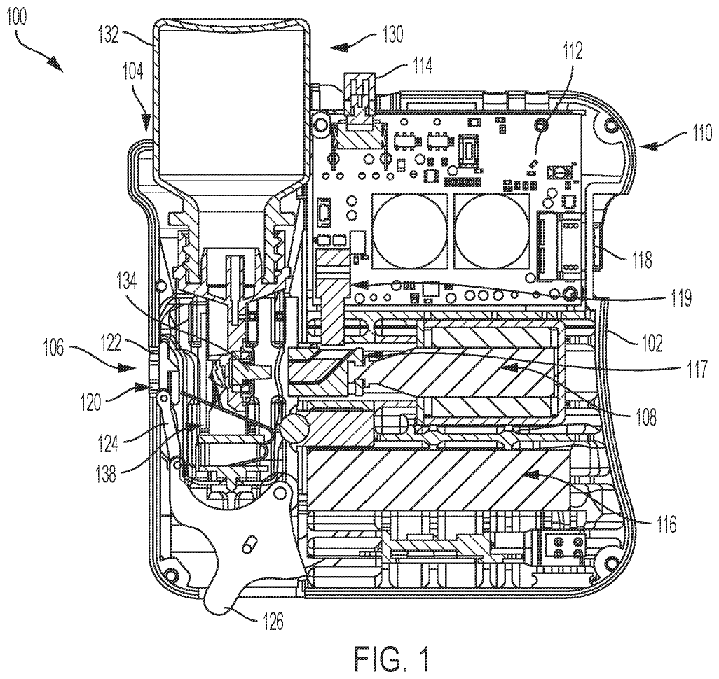

FIG. 1 is a cross-sectional view of a fluid dispensing system 100, according to aspects of the present disclosure. The system 100 includes a fluid dispenser 110 and a fluid cartridge 130 coupled to, and positioned partially within, the fluid dispenser 110. The fluid dispenser 110 includes one or more actuating mechanisms to expel a stream of fluid from the fluid cartridge 130. The fluid cartridge 130 may be a disposable component, in some embodiments. For example, the fluid cartridge 130 may include a single-use device that can be disposed after the fluid has been dispensed. As explained further below, the dispenser 110 may include one or more components for sterilizing a fluid egress of the cartridge 130 before, between, and/or after the system 100 has been used.

The fluid dispenser 110 includes a housing 102 configured to encase, enclose, and/or otherwise protect one or more dispensing components within the housing 102. The fluid dispenser 110 includes an actuating assembly 108. In some embodiments, the actuating assembly 108 may include a solenoid and a piston. In some aspects, the actuating assembly 108 may include a bistable and/or latching-type solenoid. In some aspects it may be advantageous for the solenoid to be of a bi-stable or two position latching type. A bi-stable solenoid or a latching-type solenoid may maintain both the latched and unlatched positions without constant current draw so that current is supplied only during the transition from one state to another. In addition, the force and speed and momentum of the movement with which the plunger strikes may be adjusted electronically in solenoid designs by varying the amount of current supplied. Thus, higher de-latching current may provide a higher strike force for higher viscosity liquids formulations inside the cartridge to maintain a dosage for a variety of fluid viscosities.

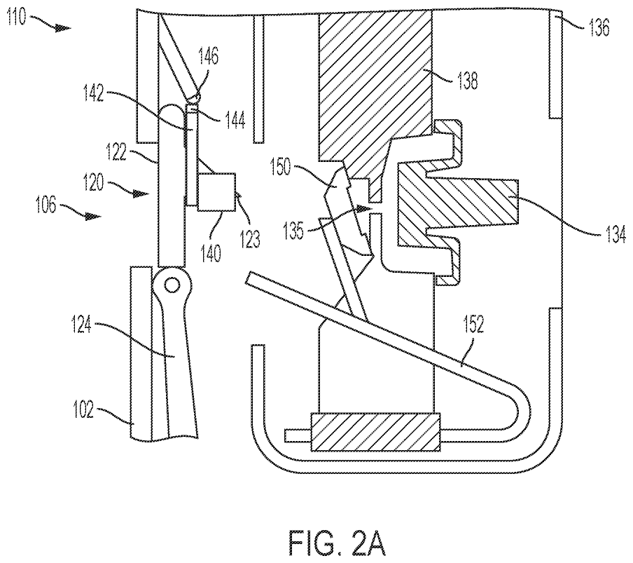

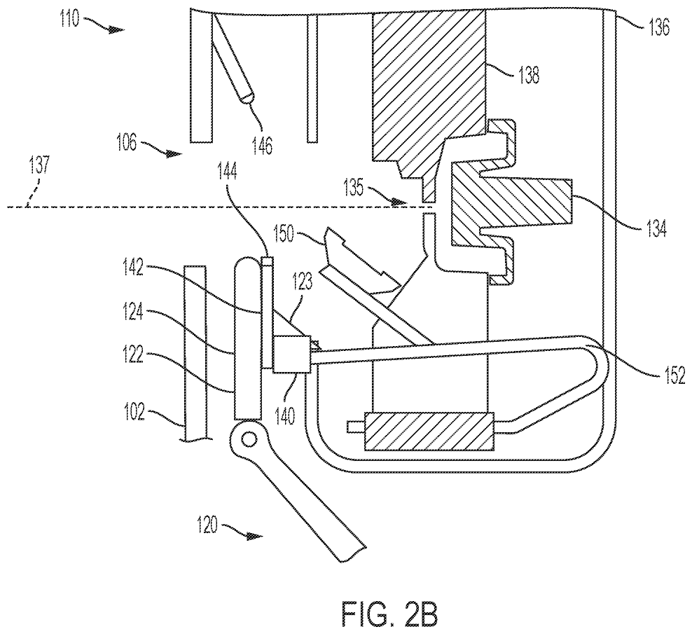

In some aspect, the solenoid may include a damper or damping feature to reduce or eliminate rebound so as not to resonate back and forth or bounce as this can draw outside air inside the nozzle opening during immediate retraction or bounce thus preventing or hindering the potential use of preservative-free formulations. Thus a solenoid with a damping feature such as a soft elastic dampener at a distal end of the solenoid to eliminate rebound motion may be used. In other aspects, the damping feature may be included on a compression membrane (see, e.g., 134, FIG. 2A) of a dispensing head. Combining the dampener with a bistable operation may allow for the outward push state to be maintained at the end of travel without any immediate retractions when current is turned off. The compression membrane can act as an internal elastic nozzle cover as well as dampener on the inside of the nozzle blocking penetration of external pathogens. For eliminating such unwanted ingress, the external nozzle cover (see, e.g., 150, FIG. 2A) may be changed to the closed position before the internal compression membrane is pulled away to return to its open or retracted position by moving the solenoid from an outward pushed state to a retracted pulled state. Thus a bistable solenoid with adjustable strike force having an end point dampener may be an advantageous configuration for a micro stream ejecting actuator. In some embodiments, the fluid cartridge further comprises an arm coupled to the polymeric cover, where the arm is movable between an open state and a closed state, and where the nozzle is exposed when the arm is in the open state.

The actuating assembly 108 may be electrically powered to impinge or strike the compression membrane 134 of the fluid cartridge 130. The fluid dispenser 110 further includes a circuit board 112 disposed within the housing 102. The circuit board 112 may include a plurality of electronic components for controlling the fluid dispensing system 100. The fluid dispenser 110 includes a user input component 114, which may include a button, coupled to the circuit board 112. By depressing the button 114, a user may initiate a fluid dispensing procedure or protocol. The protocol may be controlled by a controller or processor coupled to the circuit board 112, for example. In some embodiments, the fluid dispensing protocol may be based on input from a blink detector, a timer, a proximity sensor, and/or other sensing components. The sensing components may increase the likelihood of success that the stream of fluid reaches the eye at a desired location and profile, and in between blinks.

The dispensing device 110 includes a power supply 116. In some embodiments, the power supply 116 includes a battery. In some embodiments, the battery is a rechargeable battery. In other embodiments, the battery is a single-use or non-rechargeable battery. In some embodiments, the battery may be recharged by an interface or port 118 coupled to the circuit board 112. In some embodiments, the port 118 may provide for data communication and/or charging. The port 118 may be an industry standard port, such as universal serial bus (USB), mini-USB, micro-USB, Apple® LIGHTNING, and/or any other suitable industry standard interface or bus. Further, the power supply 116 may be controlled by one or more electronic components. In some embodiments, the power supply 116 is in communication with a processor or processing circuitry coupled to the circuit board 112. The power supply circuitry may be configured to distribute, regulate, activate, and/or perform other power supply functions to control one or more components of the device 110. For example, the power supply circuitry may supply electrical power from the power supply 116 to the actuating assembly 108 and/or nozzle sterilizing components (described further below).

The housing 102 includes or defines a first opening 104 and a second opening 106. The fluid cartridge 130 is loaded into the fluid dispenser 110 via the first opening 104. The second opening 106 is positioned adjacent to, and aligned with, a nozzle of the fluid cartridge 130. Accordingly, the second opening 106 may be positioned such that, when the fluid cartridge 130 is positioned within the fluid dispenser 110, the stream of fluid ejected from the nozzle proceeds out of the fluid dispenser 110 through the second opening 106. The housing 102 may further include one or more seating features to support and retain the fluid cartridge 130. The one or more seating features may include protrusions, ridges, and/or other geometrical elements of the housing 102 to guide the fluid cartridge 130 into place. In some aspects, the one or more seating features may be configured to assist a user in aligning the fluid cartridge 130 so that it is correctly clocked rotationally such that the nozzle is aimed outward toward the second opening 106. For example, the one or more seating features may be shaped based on an outer profile of the fluid cartridge 130.

The dispenser 110 includes a shutter assembly 120 actuatable by an actuator 126. In some aspects, the actuator 126 may be referred to as a hand activated trigger. The actuator 126 may include a manual or non-powered mechanism actuated by a user (e.g., lever, manual slide, knob, wheel, etc.) In other aspects the actuator 126 includes a powered mechanism. In the illustrated embodiment, the shutter assembly 120 includes a first shutter component 122 rotatably coupled to a second shutter component 124. The second shutter component 124 is rotatably coupled to the actuator126. In some aspects, the first shutter component 122 and the second shutter component 124 may be coupled to one another by a first hinge. In a further aspect, the second shutter component 124 and the actuator 126 may be coupled by a second hinge. In some aspects, one or more of the components 122, 124 includes a guide feature (e.g., protrusion) configured to slide along a track feature of the housing 102. For example, the shutter assembly 120 may be configured to move from a closed position in which the shutter component 122 covers or obscures the second opening 106 to an open position in which the shutter component 122 does not obscure the second opening 106 to expose the nozzle of the fluid cartridge 130. The housing 102 may include a track feature to guide the shutter assembly 120, via the guide feature, along a path leading away from the second opening 106. The first hinge coupling the first shutter component 122 and the second shutter component 124 allows the second shutter component 124 to rotate about the hinge as needed while the first shutter component 122 is moved away from the second opening 106.

The fluid cartridge 130 includes a fluid reservoir 132 configured to contain a fluid. For example, the fluid cartridge 130 may contain a pharmaceutical agent and/or other therapeutic substance. The fluid reservoir 132 is coupled to a dispensing head 138. The dispensing head 138 of the fluid cartridge 130 includes the compression membrane 134 and a nozzle (see, e.g., FIGS. 2A, 2B below). The dispensing head 138 may be configured to direct fluid in the reservoir 132 down a fluid channel and into a fluid chamber between the compression membrane 134 and the nozzle. The actuating assembly 108 may cause the fluid in the fluid chamber to expel through the nozzle, as explained further below.

In some aspects, the dispensing device 110 further includes a light source or light element 119 and a light guide 117 attached to actuating assembly 108. In some aspects, the light source 119 and light guide 117 are part of an aiming assembly for assisting the user with aiming and positioning the system 100 to dispense the fluid in a suitable area of the eye. In this regard, the light guide 117 may comprise an angled reflective surface configured to reflect and redirect light rays from the light source 119 through the membrane 134 along a dispensing axis. In this regard, the membrane 134 may be at least partially transparent such that at least a portion of light from the light source 119 reaches the user's eye. In some aspects, the light from the light source 119 may only be visible to the user when the system 100 is positioned and oriented within a suitable range for dispensing. The light source 119 may also change color or be blinked or pulsed at different rates to reflect how well the applicator is aligned to the eye using for example, a proximity sensor to sense the eye.

FIGS. 2A and 2B are cross-sectional views of the system 100, focused on the dispensing head 138 and shutter assembly 120. As explained above, the dispensing head 138 includes a compression membrane 134 and a nozzle or nozzles arrayed 135. The dispensing head 138 may further include a fluid channel extending between the fluid reservoir and a fluid chamber between the compression membrane 134 and the nozzle 135. The dispensing head 138 further includes a nozzle cover 150 configured to cover and isolate the nozzle 135 from the external environment. In some embodiments, the nozzle cover 150 includes an elastomeric material configured to create a seal with exterior surfaces of the dispensing head 138 around the nozzle 135. The nozzle cover 150 may be actuatable via a nozzle cover arm 152. The arm 152 may be flexible and/or resiliently coupled to the dispensing head 138. In some embodiments, the arm 152 is configured to flex downward to separate the nozzle cover 150 from the nozzle area. For example, the arm 152 may be configured to flex or rotate downward such that the nozzle 135 can eject the fluid stream outward through the second opening 106. The arm 152 may be actuatable by a nozzle cover releasing feature 123 coupled to the first shutter component 122, as explained further below.

The dispensing head 138 further includes a dispensing head cap 136. The cap 136 at least partially surrounds the membrane 134, the nozzle 135, the nozzle cover 150, and arm 152 thereby providing some handling protection when handling the cartridge. The cap 136 includes an opening aligned with the second opening on the opposite side. The cap 136 includes a second opening providing access to the compression membrane 134. As mentioned above, an actuating assembly may be configured to impinge or strike the compression membrane 134 of the fluid cartridge 130 to cause the fluid to between the membrane 134 and the nozzle 135 to eject via a fluid trajectory (see 137, FIG. 2B).

The shutter assembly 120 includes a nozzle cover disengaging feature 123 disposed on an inward facing surface of the first shutter component 122. The disengaging feature 123 includes a protrusion configured to catch an end of the arm 152 to disengage the nozzle cover 150 from the nozzle area of the dispensing head 138. For example, as the shutter assembly 120 moves from the closed position as shown in FIG. 2A to the open position as shown in FIG. 2B, the disengaging feature 123 may catch the end of the arm 152 to actuate the arm 152 downward. The downward movement of the arm 152 may cause the nozzle cover 150 to move away from the nozzle 135 and the trajectory 137 of fluid ejected from the nozzle 135. As explained above, the dispensing device 110 may include seating features configured to retain the fluid cartridge stationary, or substantially stationary, relative to the dispensing device 110. Accordingly, the shutter assembly 120 of the dispensing device 110 can exert a sufficient downward force on the arm 152 to flex the arm 152 downward and move the nozzle cover 150 out of the way of the trajectory 137.

Referring to FIG. 2A, the dispensing device 110 includes a sterilization component 140. In an exemplary embodiment, the sterilization component 140 is configured to emit electromagnetic radiation to kill viruses and/or bacteria in, near, and/or around the nozzle 135. In one embodiment, the sterilization component 140 includes an ultraviolet or violet light-emitting component. For example, the sterilization component 140 may include a UV LED. The sterilization component 140 is mounted to a substrate 142, which is coupled to the inward facing surface of the first shutter component 122. The sterilization component 140 is configured to emit the radiation in a direction toward the nozzle 135 when the fluid cartridge 130 is disposed within the dispensing device 110, as shown in FIGS. 2A and 2B, and when the shutter assembly 120 is in the closed position, as shown in FIG. 2A. For example, the sterilization component 140 may be positioned within or near the fluid trajectory 137 shown in FIG. 2B. Further, the sterilization component 140 may be configured to emit UV light along, parallel to, intersecting with, and/or adjacent to the fluid trajectory 137. The nozzle cover 150 may include a polymeric material that is at least partially transparent. For example, the polymeric material of the nozzle cover 150 may be at least partially transparent in the UV spectrum. In some embodiments, the nozzle cover 150 comprises a transparent thermoplastic elastomer (TPE) consisting of a transparent styrene-ethylene-butylene styrene (SEBS) or styrene-ethylene-propylene-styrene (SEPS). One example is the KRATON G1643 TPE sold by KRATON Corporation. Such materials are known to have high UV transparency and resistance to UV aging while maintaining their elasticity. Accordingly, UV light from the sterilization component 140 may travel through the nozzle cover 150 to the area around and/or inside the nozzle 135 to keep the fluid environment in and/or around the nozzle 135 sterile. In some embodiments, the nozzle cover 150 includes a transparent elastomeric and/or conformable material configured to form a seal with the surfaces around the nozzle 135.

The substrate 142 on which the sterilization component 140 is mounted may provide an electrical connection between the sterilization component 140 and a power supply (e.g., 116, FIG. 1) of the dispensing device 110. In the illustrated embodiment, the substrate 142 includes a first set of electrical contacts 144 configured to contact and electrically coupled with a corresponding set of electrical contacts 146 coupled to the housing 102 of the dispensing device 110. The substrate 142 is coupled to the shutter assembly 120 such that the substrate 142, and thereby the sterilization component 140, can receive electrical power from the power supply of the dispensing device 110 when the shutter assembly 120 is in the closed position, as shown in FIG. 2A. When the shutter assembly 120 is in the open position as shown in FIG. 2B, the electrical connection between the sterilization component 140 and the power supply is broken so that the UV component does not receive power from the dispensing device 110.

In some embodiments, the power supply of the dispensing device 110 may be configured to power the sterilization component 140 for a predetermined amount of time. For example, in response to the electrical connection formed when the shutter assembly 120 is in the closed position, the power supply of the dispensing device 110 may start a timer, and activate the sterilization component 140 for the duration of the timer. In some embodiments, the power supply may power the sterilization component 140 in periodic intervals. For example, the sterilization component 140 may be activated once per minute, once per five minutes, once per 10 minutes, once for 30 minutes, once per hour, and/or any other suitable interval. Sterilization component 140 may be activated at each interval for a configured amount of time, such as one second, five seconds, 10 seconds, 30 seconds, 60 seconds, and/or any other suitable amount of time. In other embodiments, the sterilization component 140 may be configured to activate continuously while the shutter assembly 120 is in the closed position. The time duration, intensity, frequency, and/or other parameters of the sterilization protocol may be controlled by a controller. The controller may be coupled to a circuit board, such as the circuit board 112 shown in FIG. 1.

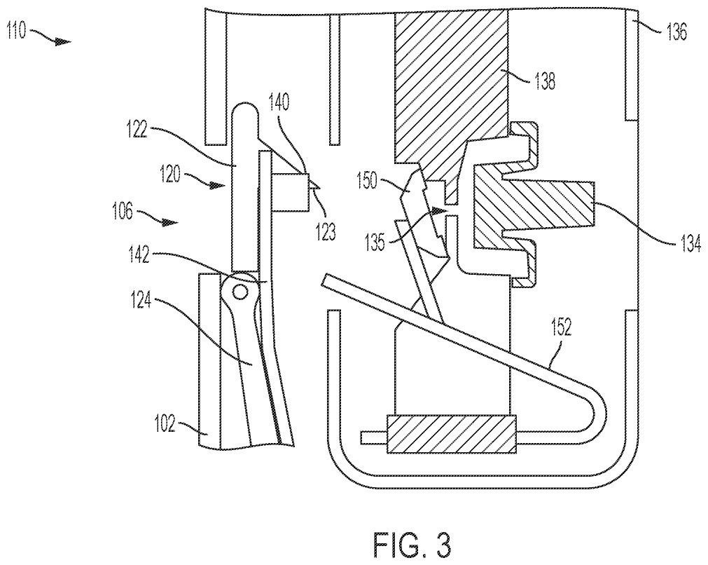

FIG. 3 is a cross-sectional view of the dispensing device 110 and the fluid cartridge 130, according to another embodiment of the present disclosure. The dispensing device 110 and the fluid cartridge 130 shown in FIG. 3 may include a plurality of components that are similar to or identical to the components of the embodiments shown in FIGS. 1-2B. For example, the dispensing device 110 includes a housing 102, a shutter assembly 120, and a sterilization component 140. The housing 102 includes or defines a second opening 106 aligned with the nozzle 135 of the fluid cartridge 130. The shutter assembly 120 includes a first shutter component 122 coupled to a second shutter component 124. The shutter assembly 120 further includes a disengagement feature 123 disposed beside the sterilization component 140. The shutter assembly 120 is configured to move from the closed position shown in FIG. 3 to an open position. The fluid cartridge 130 includes the dispensing head 138, which includes the compression membrane 134, the nozzle cover 150, and the nozzle 135. The nozzle cover 150 is coupled to the arm 152.

In the embodiment of FIG. 3, the sterilization component 140 is coupled to a flex circuit 142. The flex circuit 142 may include a flexible substrate and one or more conductive traces disposed on the flexible substrate. The flex circuit 142 may be configured to bend and/or curve with the shutter assembly 120 as the shutter assembly 120 articulates from the closed position to the open position. In some embodiments, the flex circuit 142 maintains the sterilization component 140 in electrical contact with the power supply of the dispensing device 110 continuously. In some embodiments, the dispensing device 110 may include additional sensors and/or electrical circuitry configured to trigger the sterilization component 140 to illuminate the nozzle 135 and nozzle cover 150 when the shutter assembly 120 and the nozzle cover 150 are in the closed position.

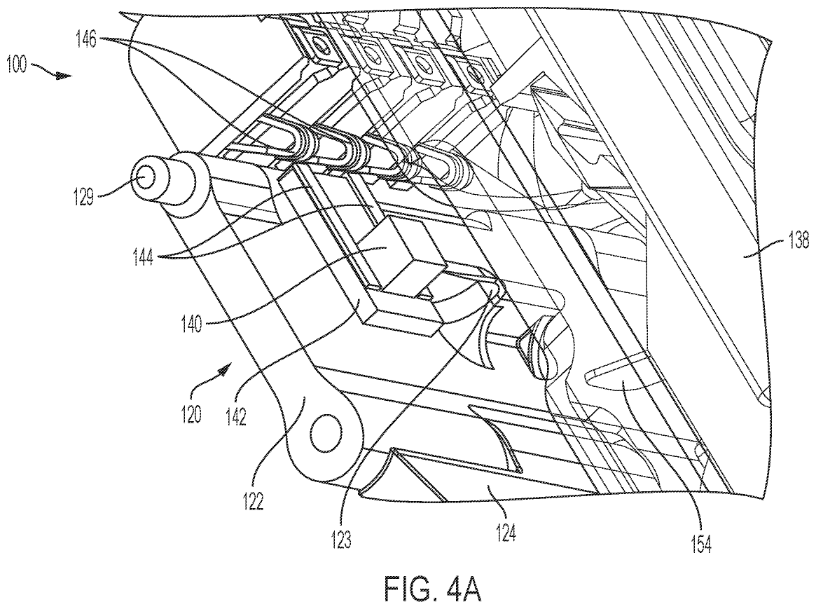

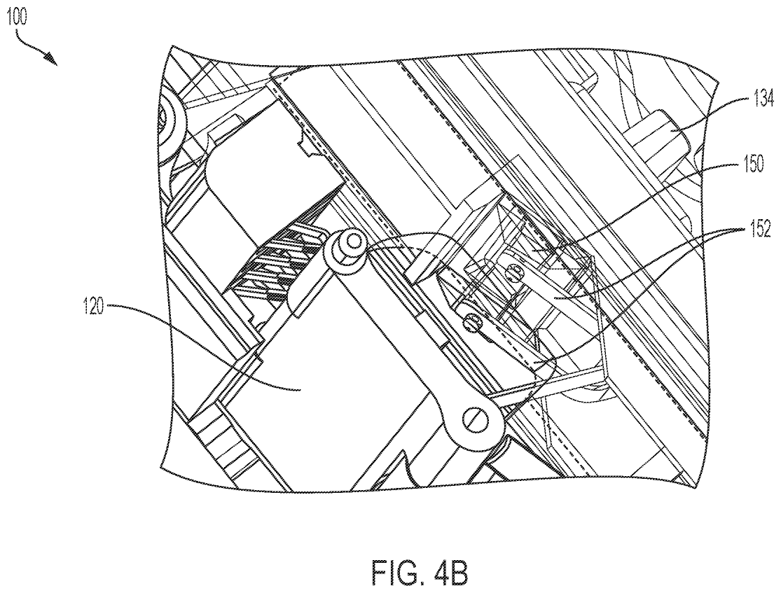

FIGS. 4A and 4B are perspective views of the shutter assembly 120 and dispensing head 138 of the system 100 shown in FIGS. 1-2B, according to an embodiment of the present disclosure. Referring to FIG. 4A, the shutter assembly 120 includes a first shutter component 122 coupled to a second shutter component 124. The first shutter component 122 and the second shutter component 124 form a hinged connection, such that the first shutter component 122 can rotate relative to the second shutter component 124 and vice versa. The first shutter component 122 includes a guide feature 129 configured to engage a track feature of the housing of the dispensing device 110. The guide feature 129 includes a round protrusion configured to fit within the track feature to maintain the shutter assembly 120 along a path defined by the track feature. The shutter assembly 120 further includes a disengagement feature 123 disposed on an inward facing surface of the first shutter component 122. The disengagement feature 123 is configured to contact a distal end 154 of a nozzle cover arm (see arm 152, FIG. 2A), and to depress the nozzle arm to disengage a nozzle cover to expose the nozzle of the dispensing head 138. The device 110 further includes a sterilization component 140 disposed on a substrate 142. The substrate 142 is coupled to the inward facing surface of the first shutter component 122. The first substrate 142 includes a set of electrical conductors 144. In some embodiments, the electrical conductors 144 may include electrical traces. For example, the set of conductors 144 may include a gold film or foil disposed on the substrate 142 in other embodiments, electrical conductors 144 may include other conductive materials, such as copper, silver, platinum, and/or alloys thereof shutter assembly 120 is shown in the closed position accordingly, the set of electrical conductors 144 is in electrical communication with a corresponding set of leaf spring electrical contacts 146. Electrical contacts 146 may be in communication with a power supply of the dispensing device 110. Accordingly the, sterilization component 140 may receive electrical power via the electrical contacts 146 when the shutter assembly 120 is in the closed position.

Referring to FIG. 4B, the nozzle cover 150 and the shutter assembly 120 are shown in the closed position. The nozzle cover arms 152 may be spring-biased to retain the nozzle cover 150 in the closed position by default. In the illustrated embodiment, the closed/open position or state of the shutter assembly 120 and the closed/open position or state of the nozzle cover 150 may be interdependent. In this regard, actuating the shutter assembly 120 to the open position may cause the nozzle cover 150 to actuate to the open position as well. Accordingly, by a single actuation of the shutter assembly 120 (e.g., via the actuator 126, FIG. 1), a user may cause both the shutter assembly 120 and the nozzle cover 150 to change between the open state and the closed state. Further, by actuating the shutter assembly 120 to the closed position, the user may cause the shutter assembly 120 and the nozzle cover 150 to change to the closed position, and for the sterilization component 140 to be activated to illuminate the nozzle cover 150 and nozzle of the dispensing head 138.

It will be understood that the system 100, including the dispensing device 110 and the fluid cartridge 130, may be changed in various ways without departing from the scope of the present disclosure. For example, although the shutter assembly 120 is described as including a plurality of shutter components rotatably coupled to one another, in other embodiments, the shutter assembly 120 may include one or more flexible pieces of material configured to curve or band, instead of or in addition to rotating relative to one another. In some embodiments, the shutter assembly 120 may be activated by an electromechanical actuating device, such as a DC motor. In some embodiments, instead of the rotating actuator 126 shown in the embodiment of FIG. 1, the dispensing device 110 may include a sliding actuator. In this regard, in some embodiments, the shutter assembly 120 may include a single shutter component configured to slide up and down between the open and closed positions. In other aspects, the rotating actuator may include an electromechanical actuator. For example, the electromechanical actuator may include a linear electromechanical actuator, an electrical motor, a solenoid, an/or any other suitable type of actuator.

In some embodiments, the fluid dispensing device 110 may include more than one sterilizing component. For example, the fluid dispensing device 110 may include an array of 2 or more micro UV LEDs. In some embodiments, the UV LEDs may include focusing optics configured to focus and/or direct UV light to a desired area on the dispensing head 138. In some embodiments, the fluid dispensing device 110 may include one or more sensors to detect whether the fluid cartridge 130 is loaded into the dispensing device 110. For example, the fluid dispensing device may be configured to activate the sterilizing components on the condition that the fluid cartridge 130 is loaded into the device 110, as indicated by the sensors. The fluid cartridge detecting sensor may include an optical sensor, mechanical sensor, magnetic, capacitive and/or any other suitable type of sensor. Further, the fluid dispensing device 110 may include wireless communication components for communicating with a mobile computing device, such as a smart phone, tablet, personal computer, and/or any other suitable wireless communication device. For example, the fluid dispensing device 110 may be configured to deliver usage data to a database available to the patient's physician. The usage data may indicate the frequency of use, dosage, the times at which the doses were delivered, and/or any other patient usage and/or compliance data.

The various embodiments described above provide several advantages. For example, by coupling the sterilizing component(s) to the shutter assembly 120, the fluid dispensing device 110 includes a simple and effective mechanism to initiate a sterilization procedure based on the closed and/or open state of the shutter assembly 120. Thus, the user can both (1) close the shutter to protect the nozzle, and (2) initiate a sterilization procedure using a single action. Further, the system 100 can contain the sterilizing radiation within the fluid dispensing device 110 and the dispensing head 138. Further, by including the electrical contacts on the shutter assembly 120 and the housing 102, the system 100 may ensure that the sterilization components 140 are only activated any time the shutter 120 is closed. Because contamination from the external environment is more likely to enter the device 110 when the shutter assembly 120 is open, it may be advantageous or beneficial to activate the sterilizing procedure via the sterilizing components 140 every time the shutter assembly 120 is closed. Further, some embodiments of the present disclosure may negate the need for additional sensors and/or complex circuitry and software to determine whether and when to active the sterilization components 140.

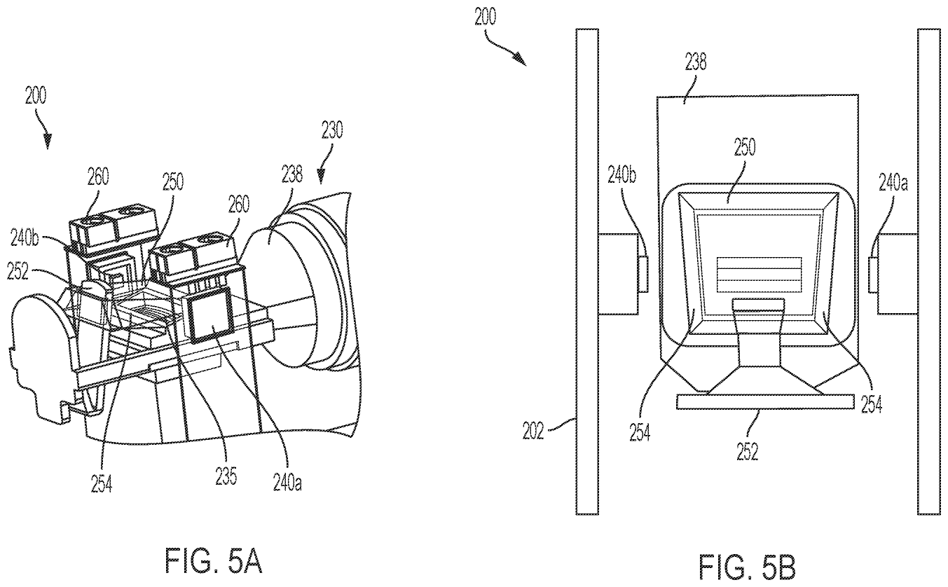

FIGS. 5A and 5B illustrate a fluid dispensing system 200 according to another embodiment of the present disclosure. FIG. 5A is a perspective view of the system 200, and FIG. 5B is a front elevation view of the system 200. In some aspects, the fluid dispensing system 200 may include one or more components similar or identical to the components of the systems 100 described above with respect to FIGS. 1-4B. For example, the system 200 includes a fluid cartridge 230 including a dispensing head 238. The system 200 may further include a fluid dispensing device including a first sterilization component 240a and a second sterilization component 240b. The sterilization components 240a, 240b may be coupled to a housing 202. For example, the sterilization components 240 may be coupled to a substrate comprising one or more electrical traces or contacts. The components 240 may be mounted to the substrate. In the illustrated embodiment, the dispensing device further includes proximity sensors 260 coupled to the substrate on which the sterilization components 240 are mounted. The proximity sensors 260 may be configured to obtain proximity measurements of the patient's eye relative to the dispensing device. The dispensing device may further include a nozzle 235 coupled to a spring-biased arm 252. The arm 252 may be shaped, sized, and/or otherwise structurally configured such that the nozzle cover 250 is in a closed position by default. In other embodiments, the nozzle cove 250 may be configured such that it is in an open position by default. The arm 252 may be actuated similarly to the arm 152 in the system 100 described above. For example, the arm 252 may be actuated by a disengaging component of a shutter assembly to move the nozzle cover 250 away from the nozzle of the dispensing head 238.

The sterilization components 240a, 240b are positioned, oriented, and configured to emit sterilizing radiation toward and through oblique surfaces 254 of the nozzle cover 250. In some aspects, the nozzle cover 250 includes a plurality of oblique surfaces oriented at nonparallel and non-perpendicular angles relative to the axes of the sterilization components 240a, 240b. The oblique surfaces 254 of the nozzle cover 250 may be configured to refract at least a portion of the sterilizing radiation (e.g., UV light) toward the nozzle 235 to sterilize the nozzle 235 and the area around the nozzle 235. Accordingly, the oblique refracting surfaces of the nozzle cover 250 in the embodiment shown in FIGS. 5A and 5B may advantageously provide a wide scattering area of the sterilizing radiation in and around the nozzle 235. The sterilization components 240a, 240b may be coupled to the dispensing device via one or more conductors. The sterilization components 240a, 240b may be in communication with a power supply of the dispensing device. The sterilization components 240a, 240b may be controlled by the dispensing device based on input and/or feedback from a sensor (e.g. proximity sensor, a Hall effect sensor, optical sensor, electrical contact) that detects when the nozzle cover 250 is in a closed position.

Referring again to FIGS. 1 and 2A, in another embodiment, the light source 119 may include a light element configured to sterilize the nozzle 135 and/or one or more components adjacent to the nozzle 135, such as the nozzle cover 150, and/or the fluid retained within the dispensing head. For example, the light source 119 may comprise a UV LED, a violet LED, and/or any other suitable light source for sterilizing the nozzle 135 and/or other components. The light guide 117 may comprise a surface reflective in at least one of the UV spectrum or the violet spectrum such that sterilizing light rays from the light source 119 are directed along the dispensing axis and toward a back side of the nozzle 135. In some aspects, the light source 119 may comprise more than one light element. For example, the light source 119 may comprise an aiming light source for positioning and orienting the dispensing device, and a sterilizing light source for sterilizing the nozzle 135, the nozzle cover 150, and/or any other component adjacent to the dispensing axis.

It will be understood that various changes, modifications, and/or substitutions can be made to the various embodiments described above without departing from the scope of the present disclosure. For example, although embodiments of the present disclosure may be described as including ultraviolet (UV) sterilizing components, it will be understood that other types of sterilizing components may be used. For example, sterilizing components in the present disclosure may be configured to emit other wavelengths of light, including visible and/or any other suitable wavelength of light. Further, although embodiments of the present disclosure may describe the sterilization components as light emitting diodes (LEDs), it will be understood that the sterilizing components may include other types of light sources, including incandescent bulbs, halogen bulbs, and/or any other suitable type of light source.

In some embodiments, the sterilization components 140 of the present disclosure may be configured to emit UVA-I and/or violet light. For example, a sterilization component (e.g., LED) may be configured to emit light having wavelengths of 340 nm-410 nm. Accordingly, the sterilization component may be configured to emit UV light, visible light, or both. In one example, the sterilization component may include a SMT405-S1 InGaN LED element manufactured by Epitex, Inc. In one exemplary embodiment, an LED may be configured to emit light having a center wavelength of 405 nm. In another example, an LED may be configured to emit light having a center wavelength of 385 nm. In some aspects, these wavelengths of light may allow some light penetration through the polymeric nozzle cover 150. For example, wavelengths ranging from 360-405 nm have sufficient penetration through SEBS TPEs to sterilize the nozzle cover 150 and the area around the nozzle cover 150, including the nozzle 135.

The sterilization components described herein may be configured to operate in a pulsed fashion. For example, a controller and/or power supply of the dispensing device may be configured to control the sterilization component to emit light with a pulsing pattern. The pulsing pattern may increase the efficiency and reduce power consumption. In some embodiments, a sterilization component may be pulsed with a pulse frequency ranging between 50 Hz-2000 Hz. In one embodiment, the sterilization component may be pulsed with a pulse frequency ranging between 100 Hz-1000 Hz. In some aspects, a pulse frequency of less than 1000 Hz, for example, may allow the sterilization component to be pulsed with a higher optical intensity and without creating excess heat. In some aspects, pulsing between 25%-40% duty cycle may advantageously increase the efficacy and/or efficiency of the sterilization protocol.

In one embodiment, the light source 119 may include a violet light LED having a wavelength between about 360 nm-410 nm. The light source 119 may be controlled by a power circuit configured to pulse the violet light as described above. In some aspects, pulsing the light source may allow for the use of higher currents without overheating or damaging the light source 119. Further, using a pulsed pattern as opposed to a continuous wave pattern may increase the effectiveness of the sterilization procedure. In this regard, some biological contaminants such as bacteria may include porphyrins which absorb visible light. In some aspects, the wavelengths of light mentioned above may target intracellular porphyrin molecules. The porphyrins may absorb the light and create free radicals that can attack both gram positive and negative bacterial walls. Accordingly, pulsing the light source may allow the porphyrins to better absorb light by giving them time to relax from an energized chemical state to a ground state, and in particular more efficiently create free radicals causing the bacterial walls to decay. It will be understood that pulsing may be used with visible or non-visible sterilizing light sources within the scope of the present disclosure.

The fluids dispensed by the devices and systems described herein may be of various forms and properties. In some embodiments, the fluids dispensed by the devices and systems of the present disclosure include therapeutic and/or pharmaceutical fluids. For example, the fluid dispensing devices and systems may be configured to provide a stream of hyaluronic acid-based eye drops to the patient's eye to treat dry eye and/or other ophthalmic conditions. In other embodiments, the fluid dispensing devices and systems may be configured to provide saline solutions to the patient's eye. Glaucoma medications may also be used. The fluid dispensing devices and systems may be used to administer a variety of pharmaceutical and/or non-pharmaceutical agents to the patient. Further, the fluids dispensed by the devices and systems may have a variety of viscosities. In some embodiments, the fluid dispensing devices and systems described herein are configured to eject a stream of a gel-like substance to the patient's eye.

Embodiments and aspects of the present disclosure, including the fluid dispensing devices and fluid cartridges, may include one or more features of the systems, devices, and/or methods described in U.S. application Ser. No. 15/931,482, filed May 13, 2020, and U.S. application Ser. No. 17/319,987, filed May 13, 2021, the entireties of which are incorporated by reference.

The phrase “at least one of A and B” should be understood to mean “A, B, or both A and B.” The phrase “one or more of the following: A, B, and C” should be understood to mean “A, B, C, A and B, B and C, A and C, or all three of A, B, and C.” The phrase “one or more of A, B, and C” should be understood to mean “A, B, C, A and B, B and C, A and C, or all three of A, B, and C.”

Generally, any creation, storage, processing, and/or exchange of user data associated with the method, apparatus, and/or system disclosed herein is configured to comply with a variety of privacy settings and security protocols and prevailing data regulations, consistent with treating confidentiality and integrity of user data as an important matter. For example, the apparatus and/or the system may include a module that implements information security controls to comply with a number of standards and/or other agreements. In some embodiments, the module receives a privacy setting selection from the user and implements controls to comply with the selected privacy setting. In other embodiments, the module identifies data that is considered sensitive, encrypts data according to any appropriate and well-known method in the art, replaces sensitive data with codes to pseudonymize the data, and otherwise ensures compliance with selected privacy settings and data security requirements and regulations.

In several example embodiments, the elements and teachings of the various illustrative example embodiments may be combined in whole or in part in some or all of the illustrative example embodiments. In addition, one or more of the elements and teachings of the various illustrative example embodiments may be omitted, at least in part, and/or combined, at least in part, with one or more of the other elements and teachings of the various illustrative embodiments.

The term “about,” as used herein, should generally be understood to refer to both numbers in a range of numerals. For example, “about 1 to 2” should be understood as “about 1 to about 2.” Moreover, all numerical ranges herein should be understood to include each whole integer, or 1/10 of an integer, within the range.

Any spatial references such as, for example, “upper,” “lower,” “above,” “below,” “between,” “bottom,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In several example embodiments, while different steps, processes, and procedures are described as appearing as distinct acts, one or more of the steps, one or more of the processes, and/or one or more of the procedures may also be performed in different orders, simultaneously, and/or sequentially. In several example embodiments, the steps, processes and/or procedures may be merged into one or more steps, processes, and/or procedures.

In several example embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims.

In the claims, any means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

What is claimed is:

1. A fluid dispenser, comprising:
a housing, defining:
a first opening configured to receive a fluid cartridge; and
a second opening positioned such that a nozzle of the fluid cartridge is aligned with the second opening when the fluid cartridge is positioned within the first opening; and
a shutter coupled to the housing and configured to articulate between a closed position and an open position, wherein the shutter is configured to obscure the second opening in the closed position and to expose the second opening in the open position, and wherein the shutter comprises a protrusion on an inward-facing surface of the shutter, the protrusion configured to engage and actuate a nozzle cover of the fluid cartridge when the shutter articulates to the open position.

2. The fluid dispenser of claim 1, wherein the shutter includes a first shutter component and a second shutter component coupled to the first shutter component by a first hinge.

3. The fluid dispenser of claim 2, further comprising a mechanical actuator coupled to the second shutter component by a second hinge, wherein the mechanical actuator is configured to move the shutter from the closed position to the open position.

4. The fluid dispenser of claim 2, further comprising:
a sterilization component coupled to the first shutter component;
a power supply electrically coupled to the sterilization component; and
a controller configured to cause the power supply to provide electrical power to the sterilization component for one or more periods of time.

5. The fluid dispenser of claim 4, wherein the sterilization component comprises a light-emitting diode (LED) configured to emit light within a range of 360 nm-410 nm.

6. The fluid dispenser of claim 1, further comprising:
a sterilization component coupled to the inward-facing surface of the shutter, wherein the sterilization component is configured to emit light toward the nozzle of the fluid cartridge when the shutter is in the closed position and the fluid cartridge is positioned within the first opening.

7. The fluid dispenser of claim 6, further comprising:
a pair of conductors coupled to the shutter and the sterilization component;
a pair of electrical spring contacts coupled to the housing, wherein the pair of electrical spring contacts are positioned to be in contact with the pair of conductors when the shutter is in the closed position; and
a power supply coupled to the housing and configured to provide electrical power to the sterilization component via the pair of electrical spring contacts.

8. A fluid dispensing system, comprising:
a fluid cartridge, comprising:
a reservoir configured to retain a fluid;
a dispensing head comprising a nozzle; and
a polymeric nozzle cover movably coupled to the dispensing head; and
an actuator assembly, comprising:
a housing configured to releasably retain the fluid cartridge, the housing comprising:
a first opening configured to receive the fluid cartridge; and
a second opening positioned adjacent the nozzle of the fluid cartridge when the fluid cartridge is disposed within the first opening;
a shutter coupled to the housing and configured to articulate between a closed position and an open position, wherein the shutter is configured to actuate the polymeric nozzle cover to expose the nozzle when the shutter is articulated from the closed position to the open position, wherein the shutter comprises an inward-facing surface, wherein the inward-facing surface faces the nozzle when the shutter is in the closed position; and
a sterilization component coupled to the inward-facing surface of the shutter.

9. The fluid dispensing system of claim 8, wherein the dispensing head further includes a compression membrane on a first side of the dispensing head, and wherein the actuator assembly further comprises a solenoid configured to strike the compression membrane to expel fluid through the nozzle.

10. The fluid dispensing system of claim 9, wherein the solenoid is at least one of a bistable solenoid or a latching-type solenoid, wherein the solenoid comprises a damping component configured to reduce rebound.

11. The fluid dispensing system of claim 9, wherein the solenoid is configured with an adjustable plunger strike force based on a value of an actuation current, wherein the strike force is electronically adjustable based on a viscosity of the fluid inside the cartridge, and wherein the solenoid is configured to dampen excess strike force to prevent rebound of the plunger.

12. The fluid dispensing system of claim 8, wherein the fluid cartridge further comprises an arm coupled to the polymeric nozzle cover, wherein the arm is movable between an open state and a closed state, wherein the nozzle is exposed when the arm is in the open state.

13. The fluid dispensing system of claim 12, wherein the shutter includes a nozzle cover disengagement feature configured to actuate the arm to the open state.

14. The fluid dispensing system of claim 12, wherein the arm is spring-biased such that the polymeric nozzle cover is in the closed state when the shutter is in the closed position.

15. The fluid dispensing system of claim 12, further comprising an electromechanical actuator coupled to the shutter, wherein the electromechanical actuator comprises a worm gear motor to actuate the shutter from the closed position to the open position and to actuate the polymeric nozzle cap from a closed position to an open position.

16. The fluid dispensing system of claim 15, wherein the linear electromechanical actuator is configured to actuate both the shutter and the polymeric nozzle cover to move from the closed state to the open state.

17. The fluid dispensing system of claim 8, further comprising:
a pair of conductors coupled to the shutter and the sterilization component;
a pair of electrical contacts coupled to the housing, wherein the pair of electrical contacts are positioned to be in contact with the pair of conductors when the shutter is in the closed position; and
a power supply coupled to the housing and configured to provide electrical power to the sterilization component via the pair of electrical contacts.

18. The fluid dispensing system of claim 17, further comprising a controller configured to cause the power supply to provide the electrical power to the sterilization component for one or more periods of time.

19. The fluid dispensing system of claim 8, wherein the polymeric nozzle cover comprises a thermoplastic elastomer that is at least partially transparent in at least one of the ultraviolet spectrum or the violet spectrum.

20. A fluid dispensing device, comprising:
a housing defining an opening, wherein the housing comprises a pair of electrical contacts adjacent to the opening;
a shutter assembly movably coupled to the housing, wherein the shutter assembly comprises:
a first shutter component; and
an electromechanical actuator coupled to the housing and the first shutter component, wherein the actuator is movable from a first position to a second position, wherein the actuator is configured to cause the shutter assembly to move from an open position to a closed position when the mechanical actuator moves from the first position to the second position; and
a sterilizing subassembly coupled to the first shutter component, wherein the sterilizing subassembly comprises:
a sterilizing light element; and
a pair of electrical conductors electrically coupled to the sterilizing light element, wherein the pair of electrical conductors are configured to contact with the pair of electrical contacts when the shutter assembly is in the closed position.
